(19) United States
(12) Patent Application Publication
(10) Pub. No.: US 2024/0177848 A1
(43) Pub. Date: May 30, 2024

(54) SERVICE RECORDS AUGMENTATION SYSTEM FOR STORING AND VIEWING A SERVICE RECORD PERTAINING TO A MEDICAL IMAGING DEVICE

(71) Applicant: KONINKLIJKE PHILIPS N.V., EINDHOVEN (NL)

(72) Inventors: Milosh STOLIKJ, EINDHOVEN (NL); Qi GAO, EINDHOVEN (NL); Mauro BARBIERI, EINDHOVEN (NL)

(21) Appl. No.: 18/515,407

(22) Filed: Nov. 21, 2023

Related U.S. Application Data
(60) Provisional application No. 63/428,455, filed on Nov. 29, 2022.

(51) Int. Cl. G16H 40/40; G06F 21/62

(52) U.S. Cl. CPC G16H 40/40; G06F 21/6245

(57) ABSTRACT

A service records system (100) includes at least one computer (102, 111) programmed to implement a service records management system (110) for storing and viewing a service record (136) pertaining to a medical imaging device (120). The service records management system includes a service record entry system (128) configured to receive and store: content entries storing content describing servicing of the medical imaging device, and control entries storing executable code (130); and a service record viewer (129) configured to display the content stored in the content entries and to control the service record viewer by executing the executable code stored in the control entries.

### CROSS REFERENCE TO RELATED APPLICATIONS

This claims the benefit of U.S. Provisional Patent Application No. 63/428,455 filed Nov. 29, 2022. This application is hereby incorporated by reference herein.

### FIELD

The following relates generally to the medical device maintenance arts, medical imaging device maintenance arts, medical device maintenance scheduling arts, and related arts.

### BACKGROUND

Medical devices occasionally exhibit a malfunction, which requires maintenance. Depending on the malfunction, the problem can sometimes be handled remotely, i.e., by a remote service engineer (RSE). In other cases, the remote service engineer can issue a request for a Field Service Engineer (FSE) to repair the medical device on-site. One of the sources of information that engineers inspect are log files from medical devices. They are inspected at various stages of the problem handling, for finding clues or indications that identify the root cause of the problem. All engineers capture the actions they take, and any findings, in textual format, as part of service records. Most frequently, the service records are a mixture of structured and semi-structured text.

The service records are used for multiple purposes. Frequently, during the resolution of a malfunction, multiple people may be involved. For example, one RSE may start the diagnostic, before it is escalated to another (more senior/skillful) RSE. In another example, like explained before, an RSE may require an FSE to follow up with the diagnostic on site. In these situations, the exchange of information between the engineers may take place via service records. Service records are used to improve maintenance processes, e.g., by mining for best practices. Service records are used by R&D organizations for reliability analysis of the devices.

Service engineers are under pressure while resolving malfunctions. Therefore, they may not be able to capture all details of the actions they take and their findings. Furthermore, log files can be large, and take a long time to download and analyze, which increases the total diagnostic and repair time. However, not everything from the log files is useful at different stages of the diagnostic process. Some of the information needs to be processed before it becomes useful (e.g., different log files need to be aligned in time, measurement readings need to be investigated only if over a certain threshold etc.). As a result, currently, small amount of information from log files is manually extracted by RSEs and logged in service records (i.e., a manual copy/paste action).

The following discloses certain improvements to overcome these problems and others.

### SUMMARY

In one aspect, a service records system includes at least one computer programmed to implement a service records management system for storing and viewing a service record pertaining to a medical imaging device. The service records management system includes a service record entry system configured to receive and store content entries storing content describing servicing of the medical imaging device, and control entries storing executable code; and a service record viewer configured to display the content stored in the content entries and to control the service record viewer by executing the executable code stored in the control entries.

In another aspect, a service records maintenance method includes receiving and storing a service record of a medical imaging device including content entries storing content describing servicing of the medical imaging device, and at least one control entry storing executable code; displaying content stored in the content entries of the service record on a computer; and controlling the displaying by executing, by the computer, the executable code stored in the at least one control entry of the service record of the medical imaging device.

In another aspect, a non-transitory computer readable medium stores instructions readable and executable by at least one computer to receive and store as service record of a medical imaging device including: content describing servicing of the medical imaging device, and at least one control entry of the service record of the medical imaging device which comprises executable code; and view the service record of the medical imaging device including executing the executable code of the at least one control entry to control the view of the service record.

One advantage resides in ensuring all details are captured during a servicing session of a medical device by a SE.

Another advantage resides in finding relevant details for a servicing session in log files of a medical device undergoing the servicing session.

Another advantage resides in reducing a repair time of a medical device.

Another advantage resides in reducing copy/paste actions by a SE of log file information during a servicing session of a medical device.

Another advantage resides in providing a service records maintenance system that controls access to sensitive information such as intellectual property stored in the system.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system for servicing a medical device in accordance with the present disclosure.

FIG. 2 shows exemplary flow chart operations of the system of FIG. 1.

FIGS. 3-7 show other embodiments of the system of FIG. 1.

### DETAILED DESCRIPTION

The following relates to: (i) a way to make it more efficient for a service engineer (SE) to enter service call data and processed log files into the service record; and (ii) a way to protect intellectual property (IP) that may be contained in the service record.

As background, a customer relationship management (CRM) system, ticketing system, or the like is commonly used by SE's during service calls, and the CRM system provides a user interface (UI) for entering service call data, including for example: automatically generated machine log messages, test images, results of data analyses performed by the SE, a record of the sequence of actions taken by the SE, annotations to data made by the SE, and so forth. This information is currently entered into the service record mostly by cutting/pasting content. The resulting service record becomes associated with the imaging device—hence, any SE from any service provider (the imaging device vendor or third-party service provider, and both remote SEs and on-site field SEs) has full access to the service record.

To make entry of service call data more efficient, the following discloses creating a new type of service record entry, referred to herein as a control entry, which contains executable instructions. The control entry contains executable code such as Python code or R code, which is executable by an interpreter. (Storing compiled machine code or intermediate bytecode is also contemplated if the control entry is encapsulated so it does not need to contain only text). The instructions contained in a control entry can be generated manually by the SE, or automatically by a set of scripts or other software that monitors the SE interactions with the machine log and other data sources and generates the instructions and stores them in a control entry or entries of the service record. The service record viewer component of the CRM system is also modified to handle a control entry of the service record by piping the instructions contained in the control entry to a suitable interpreter (e.g. a Python interpreter) or other code-execution environment such as a Java virtual machine or the microprocessor itself in the case of compiled code, and receiving and inserting the output of the executed instructions as part of the displayed service record.

In some embodiments, suitable application program interface (API) components are provided to capture and incorporate data from various data analysis software applications, such as machine log message data viewing and sorting software, the imaging device controller (to capture test images), and/or so forth. Additionally, if the system automatically records sequences of actions performed by the SE, these could be filtered to remove sequence portions that led to “dead ends,” so that the recorded sequence most efficiently reaches the solution. The “dead ends” can be identified manually by asking the SE to confirm or reject inclusion of the use of each file in the service record or can be identified automatically based on whether the content of a given file was actually used in resolving the service call.

In a further aspect, IP protection is supported. As previously noted, conventionally, any SE from any service provider has full access to the service record. This can be problematic if the service record includes entries made by a service engineer of one entity (e.g., the imaging device vendor) that should not be available to a service engineer of another entity (e.g., a third party service provider). Consequently, it may be advantageous to limit, obfuscate, or encrypt information included in the service record to avoid inadvertent disclosure of trade secrets, proprietary diagnostic techniques, or other IP.

To achieve this, the following discloses including an IP protector component in the CMS, which polices information being added to the service record. This aspect is facilitated by the disclosed control entries of the service record, since the instructions contained in a control entry can include executable code operative to obfuscate or encrypt or otherwise limit dissemination of information stored by the “instructions” entity. Initially, the executable code of such a control entry analyzes content to assign a protection level to the various pieces of information. The instructions can be hard-coded based on a priori identification of diagnostic methods and information sources that are desired to be protected.

In one approach, when the IP protector identifies that the SE is entering protected content into the service record, the SE is warned of this (and optionally is prevented from entering the information into the service record at all).

In another approach, protected content can be obfuscated by adding predefined changes such as adding additional information sources that are not actually used by the SE in the analysis, changing values of time offsets used in the data analysis or the like. In yet another approach, the protected content can be encrypted.

In the latter two cases, the service record viewer of the CMS system used by the service provider represented by the SE entering the data into the service record is modified to de obfuscate or decrypt the protected content when another SE is subsequently viewing the service record. To enable de-obfuscation when appropriate, the obfuscating data additions or modification should be added in a deterministic (but complex and difficult to recognize) manner so as to be reversible. In some embodiments, different Customer Relationship Management (CRM) system users may have different protection levels (e.g. for an SE who is an employee of the vendor may have full access, while a third-party SE who is contracted by the vendor to provide servicing may have limited access), and the amount of de-obfuscation or the portion of the content that is decrypted may vary based on the consuming SE's protection level. On the other hand, a third-party SE unaffiliated with the vendor will typically use a different CMS (or at least a CMS that is not modified to include the IP protector as disclosed herein) and hence will have no access to the protected content.

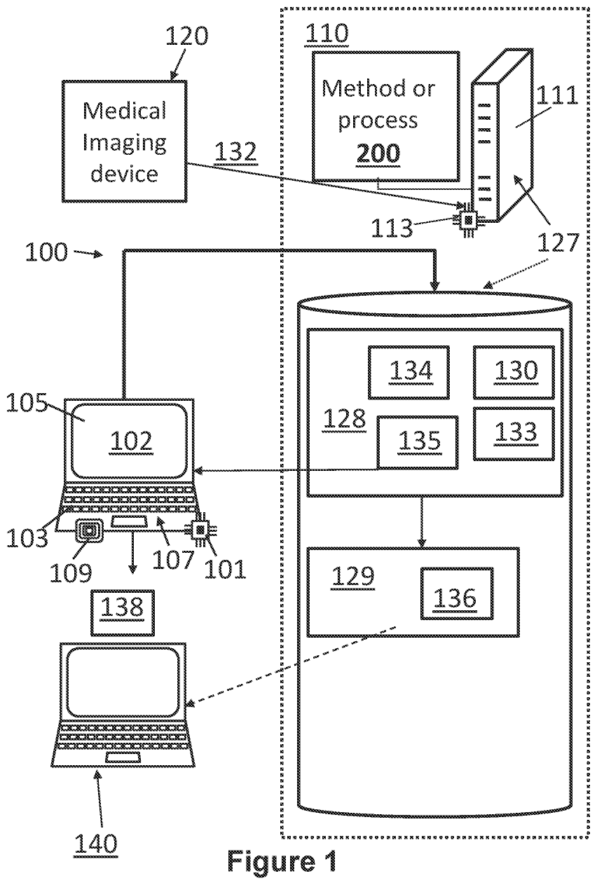

With reference to FIG. 1, an illustrative service records system 100 for supporting a service engineer in servicing a device 120 (e.g., a medical imaging device—also referred to as a medical device, an imaging device, imaging scanner, and variants thereof) is diagrammatically shown. By way of some non-limiting illustrative examples, the medical imaging device under service may be a magnetic resonance imaging (MRI) scanner, a computed tomography (CT) scanner, a positron emission tomography (PET) scanner, a gamma camera for performing single photon emission computed tomography (SPECT), an interventional radiology (IR) device, or so forth. (More generally, the disclosed approach can be applied in conjunction with any type of computerized device that automatically generates log data that are analyzed by predictive models to predict component failures, e.g., the approach could be applied to a commercial airliner, radiation therapy device, or so forth). As shown in FIG. 1, the service records system 100 includes, or is accessible by, a service device 102 that may for example be a workstation or electronic processing device used by an FSE. The service device 102 may for example be a portable device such as a notebook computer that is carried or accessed by an FSE. The service device 102 can be a desktop computer or a personal device, such as a mobile computer system such as a laptop or smart device. In other embodiments, the service device 102 may be an imaging system controller or computer integral with or operatively connected with the imaging device undergoing service (e.g., at a medical facility). As another example, the service device 102 may be a portable computer (e.g., notebook computer, tablet computer, or so forth) carried by a FSE performing diagnosis of a fault with the imaging device and ordering of parts. In another example, the service device 102 may be the controller computer of the imaging device under service, or a computer based at the hospital. In other embodiments, the service device 102 may be a mobile device such as a cellular telephone (cellphone) or tablet computer.

The service device 102 includes a display 105 via which alerts generated by predictive failure models are displayed, along with likely root cause and service action recommendation information as disclosed herein. The service device 102 also preferably allows the service engineer to interact with the servicing support system via at least one user input device 103 such a mouse, keyboard, or touchscreen. The service device further includes an electronic processer 101 and non-transitory storage medium 107 (internal components which are diagrammatically indicated in FIG. 1). The non-transitory storage medium 107 stores instructions which are readable and executable by the electronic processor 101 for interfacing with the service records system 100. The service device 102 also includes a communication interface 109 to communicate with a backend server or processing device 111, which typically implements the computational aspects of the service records system 100 (e.g., the server 111 has the processing power for implementing computationally complex aspects of the service records system 100). Such communication interfaces 109 include, for example, a wired and/or wireless Ethernet interface (e.g., in the case in which the service device 102 is a RSE workstation); or in the case in which the service device 102 is a portable FSE device the interface 109 may be a wireless Wi-Fi or 4G/5G interface or the like for connection to the Internet and/or an intranet. Some aspects of the service records system 100 may also be implemented by cloud processing or other remote processing (that is, the server computer 111 may be embodied as a cloud-based computing resource comprising a plurality of interconnected servers).

In some embodiments, the service records system 100 can include a second service device 140 operable by a RSE at a location remote from the device 120. The second service device 140 includes substantially the same components as the service device 102 and will not be repeated for brevity.

In illustrative FIG. 1, the servicing support system further includes a backend 110 (e.g., implemented and/or owned by the imaging device vendor or leased by the vendor from a cloud computing service provider). The backend 110 receives log data (e.g., a machine log automatically generated by the medical imaging device 120, a service log for the medical imaging device 120, and/or so forth) on a continuous or occasional basis (e.g., in some setups the imaging device 120 uploads machine log entries to the backend 110 on a daily basis). The backend may optionally perform processing for performing predictive fault modeling, in which case an issue may be opened automatically (e.g., if fault modeling predicts an X-ray tube will likely fail in the next month, a service ticket may be automatically generated to replace the X-ray tube). The backend may optionally additionally or alternatively provide a ticketing system or the like via which users can manually open a ticket for an open issue.

The backend 110 optionally further performs maintenance service analyses on the backend server 111, which is equipped with an electronic processor 113 (diagrammatically indicated internal component). These analyses provide automated tracking of the handling of an automatically or manually opened issue. The server 111 is equipped with non-transitory storage medium 127 (internal components which are diagrammatically indicated in FIG. 1). While a single server computer is shown, it will be appreciated that the backend 110 may more generally be implemented on a single server computer, or a server cluster, or a cloud computing resource comprising ad hoc-interconnected server computers, or so forth. Furthermore, while FIG. 1 shows a single medical imaging device 120, more generally the backend 110 will receive log data from many medical imaging devices (e.g., tens, hundreds, or more imaging devices) and performs the disclosed processing for each such medical imaging device.

With continuing reference to FIG. 1, the non-transitory computer readable medium 127 stores instructions readable and executable by the server 111 to implement a service record entry system 128 configured to receive and store control service record entries, including both content entries that store information entered by the service engineer, and (as disclosed herein) further including control entries storing executable code comprising machine-readable code or commands 130. The control entries can store code for performing various functions, such as for processing content entries storing content data 132 generated by the imaging device 120 (e.g., log data, configuration data, imaging data, and so forth), and/or processing content entries (e.g., to implement IP protection by encrypting or obfuscating portions of the content entries that contain trade secrets or other IP). The machine-readable commands 130 can be generated by the SE before or during the servicing session and stored in the non-transitory computer readable medium 127. In some examples, the machine-readable commands 130 can comprise Python code, R code, or any other suitable language, and/or can comprise Java virtual machine-executable byte-code, and/or compiled code directly executable by the microprocessor 113 of the server 111. In another example, the machine-readable commands 130 can be generated automatically by the service records management system 100.

The non-transitory computer readable medium 127 also stores a service record viewer 129 configured to display the content stored in the content entries of the service record, and to control the service record entry system 128 by executing the executable code 130 stored in the control entries of the service record. In some embodiments, the service record entry system 128 is configured to receive and store control entries including the log data processing entries configured to store executable instructions operating to process the log data 132 generated and stored by the medical imaging device 120. For example, the service record entry system 128 is configured to automatically generate a log data processing entry by capturing user inputs received from a user interacting with the log data 132 (i.e., via the FSE service device 102 and/or the RSE service device 140). To do so, the service record entry system 128 is configured to automatically generate the log data processing entry by further filtering the user inputs received from the user interacting with the log data 132 to remove user inputs that did not contribute to the servicing of the medical imaging device 120. The service record viewer 129 is configured to execute the executable code stored in a log data processing entry and to display the processed log data output by the execution of the executable code 130 on a graphical user interface (GUI) 138 presented by the service record viewer 129 of the FSE service device 102 and/or the RSE service device 140.

In another embodiment, the service record entry system 128 is configured to receive and store control entries including imaging test entries 133 configured to store executable instructions operating to control the medical imaging device 120 to acquire a test image. The service record viewer 129 is configured to execute the executable code 130 stored in an imaging test entry and to display the test image acquired by the medical imaging device 120 controlled by the execution of the executable code 130 on the GUI 138 of the FSE service device 102 and/or the RSE service device 140.

In another example embodiment, the service record entry system 128 also stores control entries including application program interface (API) entries 134 for generating and/or collecting data related to the servicing session. The API entries 134 can include various data analysis software applications, such as machine log message data viewing and sorting software, the imaging device controller (to capture test images), and/or so forth. The API entries 134 are configured to store executable instructions 130 operating to invoke an application program to process the log data 132 generated and stored by the medical imaging device 120. The service record viewer 129 is configured to execute the executable code 130 stored in an API entry 134 to invoke or call an application program (and optionally to pass information by way of API call parameters in accord with the API call format) and to receive and display the processed log data 132 output by the application program invoked by the execution of the executable code 130 on the GUI 138 of the FSE service device 102 and/or the RSE service device 140.

In another example embodiment, the service record entry system 128 is configured to receive and store control entries including access-limiting entries 135 configured to store executable instructions 130 operative to limit access to protected content of service records based on a user access level. The service record viewer 129 is configured to execute the executable code stored in an access-limiting entry 135 to control the service record viewer 129 to limit the display of the protected content of the service record of the medical imaging device 120 based on the user access level on the GUI 138 of the FSE service device 102 and/or the RSE service device 140. In one example, the service record viewer 129 is configured to execute the executable code 130 stored in the access-limiting entry 135 to prevent the service record viewer 129 from displaying the protected content of a service record 136 of the medical imaging device 120 based on the user access level. In another example, the service record entry system 128 is configured to obfuscate or encrypt the protected content, and the service record viewer 129 is configured to execute the executable code 130 stored in the access-limiting entry 135 to cause the service record viewer 129 to de-obfuscate or decrypt the protected content of the service record 136 of the medical imaging device 120 only if the protected content is accessible to the user access level.

The non-transitory storage medium 127 further stores instructions executable by the electronic processor 113 of the backend server 111 to perform a service records maintenance method 200 for a current service case for maintenance of the medical imaging device 120.

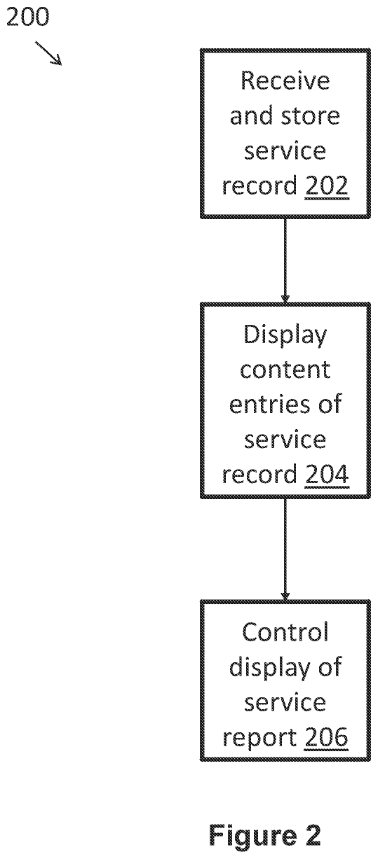

With continuing reference to FIG. 1 and further reference to FIG. 2, an illustrative embodiment of the method 200 executable by the electronic processor 113 is diagrammatically shown as a flowchart. In some examples, the method 200 may be performed at least in part by cloud processing.

At an operation 202, a service record 136 of a medical imaging device 120 including content entries 130 storing content describing servicing of the medical imaging device 120 is received and stored in the service record entry system 128. In some examples, the receiving and storing of the service record 136 includes obfuscating or encrypting the protected content of the service record 136 of the medical imaging device 120.

At an operation 204, content stored in the content entries 130 of the service record 136 are displayed on a computer (i.e., on the GUI 138 of the FSE service device 102 and/or the RSE service device 140).

At an operation 206, the displaying of the service record 136 is controlled by executing the executable code 130 stored in the at least one control entry of the service record 136 of the medical imaging device 120. This can be performed in a variety of manners. In one example, the controlling operation includes executing the executable code 130 stored in the at least one control entry to process the log data 132 generated and stored by the medical imaging device 120 and to display the processed log data 132. In another example, the executable code 130 stored in the at least one control entry is executed to control the medical imaging device 120 to acquire a test image and to display the test image. In another example, the executable code 130 stored in the at least one control entry is executed to invoke an API 134 to process the log data 132 and to display an output of the invoked API 134 (i.e., on the GUI 138 of the FSE service device 102 and/or the RSE service device 140).

In a particular embodiment, the controlling operation 206 includes executing the executable code 130 stored in the at least one control entry to limit the displaying of protected content of the service record 136 based on a user access level. The displaying of the protected content based on the user access level can then be prevented. For example, the executing of the executable code 130 to limit the displaying of protected content includes de-obfuscating or decrypting the protected content of the service record 136 only if the protected content is accessible to the user access level.

### EXAMPLES

Instead of copy/pasting individual parts of (multiple) log files, the service records system 100 is configured to store the machine-readable commands 130 for processing log data 132, within the service records 136. The machine-readable commands 130 can be written manually, by the SE, or automatically, by monitoring software of the service records system 100. The machine-readable commands 130 are parsed by an interpreter component, to re-construct the relevant information from the raw log data. That enables a more efficient and more complete information exchange, for another service engineer to continue with diagnostic/troubleshooting.

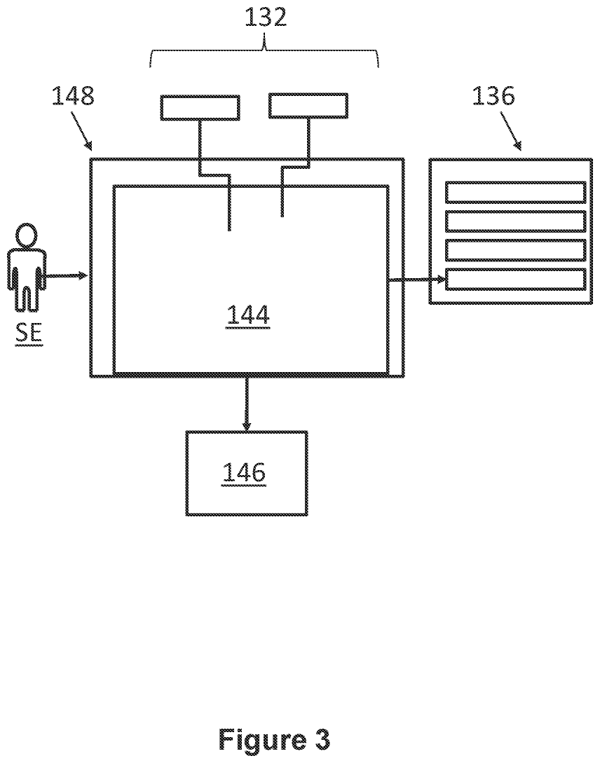

FIG. 3 shows an example of the manual capturing of machine-readable commands 130 as a jupyter notebook 144 (https://jupyter.org/) that a SE uses to load, analyze and summarize the log data. Within the notebook 144, the SE uses a common programming language (typically python or R) for data manipulation. The jupyter notebook 144 is then stored as part of the service record 136 (e.g., as a separate record type). The generated images and summaries can be extracted and stored in another format (e.g., JPG for images, plain text for SE comments). The implementation of an interpreter component 146 is then a jupyter server 148, with an appropriate interpreter for the selected programming language (i.e., python or R).

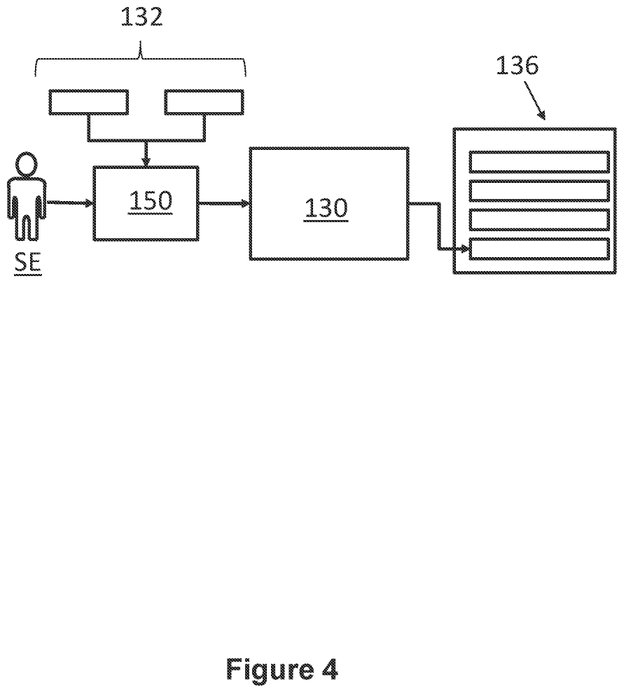

FIG. 4 shows an example of the automated capturing of machine-readable commands 130. Here, the SEs use additional tools for analyzing the log data. Selected actions taken by the SE within the tool, are stored as instructions. These can be replayed, in a separate interpreter component 146 or within a log viewer 150, to reproduce the same place where the engineer was in analyzing the log files. A non-exhaustive list of actions that may be saved are, for example, loading and closing files, open/close file instructions, selection of log lines, highlight specific line numbers, copy part of log file, highlight specific line numbers, open two files next to each other, correct timing in two log files, search through log file, filter log file by keyword/expression, and so forth.

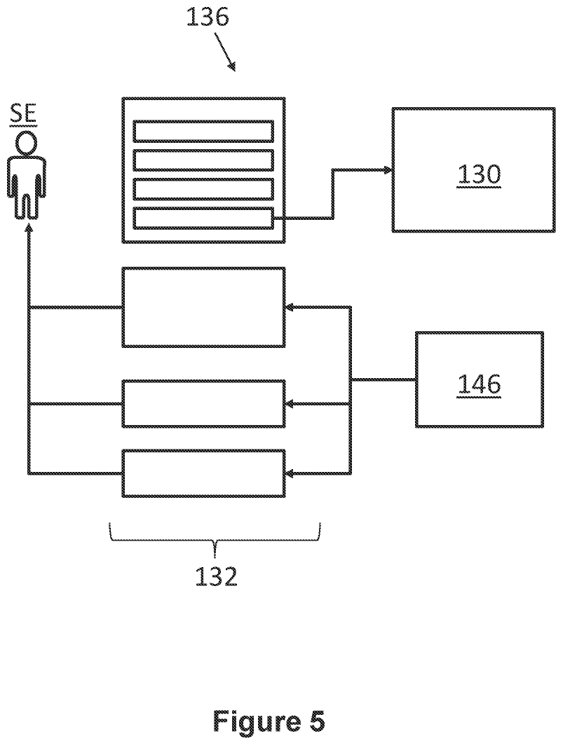

The instructions stored within a service record can be used by other SEs. This can be an engineer working on the same service case (e.g., an FSE), or a SE working on another (similar) case (e.g., an RSE), who is trying to resolve a similar problem in another device. In both scenarios, the SEs use the interpreter component 146 to load the machine-readable commands 130, and retrieve the performed analysis, as shown in FIG. 5. The interpreter component 146 may retrieve the necessary log data 132 for the analysis, or invoke third-party tools, such as the log viewer 150, as specified in the machine-readable commands 130.

In some embodiments, the machine-readable commands 130 may include two parts: metadata and executable code. The metadata describes who performed the analysis, what log data was analyzed, the protection level of the analysis (e.g., proprietary, proprietary but can be shared with partners or open), and which actions were performed. An example of a data model for the metadata is shown in Table 1.

TABLE 1
Structure of metadata in machine-readable commands 130

| Field | Type | Description |
|---|---|---|
| StartTime | Timestamp | When did the analysis take place |
| UserID | Text | Which user performed the analysis |
| UserProfile | Categorical | User type (e.g., Remote Service |
| | | Engineer, Field Service Engineer, |
| | | Contractor etc.) |
| AnalysisType | Categorical/ | What type of analysis was being |
| | Text | performed (e.g., summarization, |
| | | filtering, merging data across log files |
| | | etc.) |
| ProtectionLevel | Categorical | What type of access is required to view |
| | | the analysis. |
| LogData | Structured | Metadata of the log data being analyzed. |
| | | Depends per implementation, e.g., can |
| | | include name of file, type of file (plaint |
| | | text/xml), line numbers etc. |

In some embodiments, knowing which parts of the data 132 are relevant to troubleshoot certain cases is often protected by trade secrets or other IP. SEs of different service organizations may have to interact with each other to solve cases. For example, SEs of original equipment manufacturers (OEMs) may need to interact with SEs from independent-service-providers or with biomedical engineers at hospitals. During these interactions, it is important that enough information is exchanged to solve cases, while preserving intellectual property (IP) and preventing violations of trade secrets.

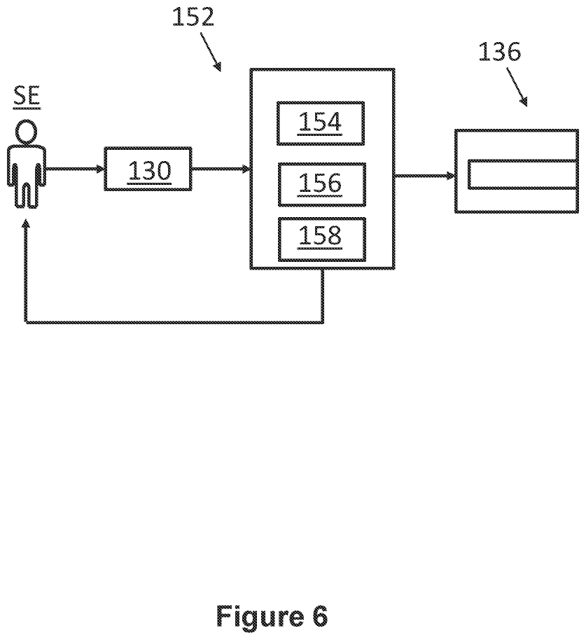

To do so, the service records system 100 includes an IP protector component 152 (FIG. 6) configured to transform the machine-readable commands 130 into a format suitable for sharing with third parties. The IP protector component 152 adds instructions to select and view and manipulate device log data 132 that have nothing to do with the actual case at hand. An obfuscation component 154 is configured to obfuscate the actual data 132 and process that one SE has followed to troubleshoot, while still providing the result. For example, if a data from a certain time interval is needed, the obfuscation component 154 can enlarge the interval or select multiple intervals such that the SE receiving the machine-readable commands 130 may not know for sure which time interval is the one determining the final service action.

In some embodiments, if one logged error is used to deduce a certain root cause and trigger a certain service action, the obfuscator component 154 can add instructions to select additional random errors, masking therefore which error is responsible for the planned actions.

In some embodiments, the IP protect component 152 includes a privacy analyzer component 156 configured to detect events and data in the log data 132 that is protected by trade secret and warns the SE not to communicate the information any further.

In another embodiment, the IP protector component 152 includes an encryption component 158 configured to encrypt the sensitive information so that only SEs with sufficient entitlements can see it.

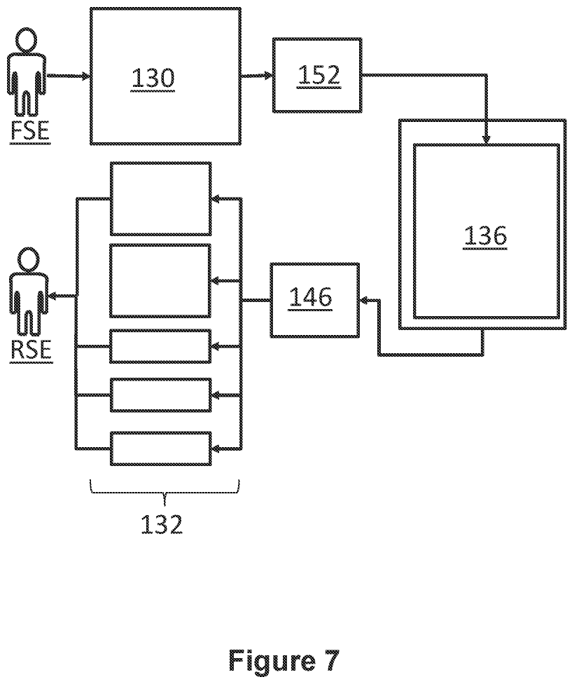

Another example of the service records system 100 is shown in FIG. 7. The IP protector component 152 analyses the machine-readable commands 130 to determine if any confidential data should be deleted or removed. The service report 136 is then generated by an FSE. A RSE can then retrieve and view the completed service record 136, along with selected portions of the log data 132 (with the confidential data removed). The RSE can then update the service report 136 or assist the FSE with the servicing session.

A non-transitory storage medium includes any medium for storing or transmitting information in a form readable by a machine (e.g., a computer). For instance, a machine-readable medium includes read only memory (“ROM”), solid state drive (SSD), flash memory, or other electronic storage medium; a hard disk drive, RAID array, or other magnetic disk storage media; an optical disk or other optical storage media; or so forth.

The methods illustrated throughout the specification, may be implemented as instructions stored on a non-transitory storage medium and read and executed by a computer or other electronic processor.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A service records system, comprising:
at least one computer programmed to implement a service records management system for storing and viewing a service record pertaining to a medical imaging device, the service records management system including:
a service record entry system configured to receive and store: content entries storing content describing servicing of the medical imaging device, and control entries storing executable code; and
a service record viewer configured to display the content stored in the content entries and to control the service record viewer by executing the executable code stored in the control entries.

2. The service records system of claim 1, wherein:
the service record entry system is configured to receive and store control entries including log data processing entries configured to store executable instructions operating to process log data generated and stored by the medical imaging device; and
the service record viewer is configured to execute the executable code stored in a log data processing entry and to display the processed log data output by the execution of the executable code.

3. The service records system of claim 2, wherein the service record entry system is configured to automatically generate a log data processing entry by capturing user inputs received from a user interacting with the log data generated and stored by the medical imaging device during servicing of the medical imaging device.

4. The service records system of claim 3, wherein the service record entry system is configured to automatically generate the log data processing entry by further filtering the user inputs received from the user interacting with the log data generated and stored by the medical imaging device during the servicing of the medical imaging device to remove user inputs that did not contribute to the servicing of the medical imaging device.

5. The service records system of claim 1, wherein:
the service record entry system is configured to receive and store control entries including imaging test entries configured to store executable instructions operating to control the medical imaging device to acquire a test image; and
the service record viewer is configured to execute the executable code stored in an imaging test entry and to display the test image acquired by the medical imaging device controlled by the execution of the executable code.

6. The service records system of claim 1, wherein:
the service record entry system is configured to receive and store control entries including application program interface (API) entries configured to store executable instructions operating to invoke an application program to process log data generated and stored by the medical imaging device; and
the service record viewer is configured to execute the executable code stored in an API entry and to display the processed log data output by the application program invoked by the execution of the executable code.

7. The service records system of claim 1, wherein:
the service record entry system is configured to receive and store control entries including access-limiting entries configured to store executable code operative to limit access to protected content of service records based on a user access level; and
the service record viewer is configured to execute the executable code stored in an access-limiting entry to control the service record viewer to limit the display of the protected content of the service record of the medical imaging device based on the user access level.

8. The service records system of claim 7, wherein the service record viewer is configured to execute the executable code stored in the access-limiting entry to prevent the service record viewer from displaying the protected content of the service record of the medical imaging device based on the user access level.

9. The service records system of claim 7, wherein:
the service record entry system is configured to obfuscate or encrypt the protected content; and
the service record viewer is configured to execute the executable code stored in the access-limiting entry to cause the service record viewer to de-obfuscate or decrypt the protected content of the service record of the medical imaging device only if the protected content is accessible to the user access level.

10. A service records maintenance method, comprising:
receiving and storing a service record of a medical imaging device including content entries storing content describing servicing of the medical imaging device, and at least one control entry storing executable code;
displaying content stored in the content entries of the service record on a computer; and
controlling the displaying by executing, by the computer, the executable code stored in the at least one control entry of the service record of the medical imaging device.

11. The service record maintenance method of claim 10, wherein the controlling includes:
executing the executable code stored in the at least one control entry to process log data generated and stored by the medical imaging device and to display the processed log data.

12. The service record maintenance method of claim 10, wherein the controlling includes:
executing the executable code stored in the at least one control entry to control the medical imaging device to acquire a test image and to display the test image.

13. The service record maintenance method of claim 10, wherein the controlling includes:
executing the executable code stored in the at least one control entry to invoke an application program to process log data generated and stored by the medical imaging device and to display an output of the invoked application program.

14. The service record maintenance method of claim 10, wherein the controlling includes:
executing the executable code stored in the at least one control entry to limit the displaying of protected content of the service record of the medical imaging device based on a user access level.

15. The service record maintenance method of claim 14, wherein the executing of the executable code stored in the at least one control entry to limit the displaying of protected content includes:
preventing the displaying of the protected content based on the user access level.

16. The service record maintenance method of claim 14, wherein:
the receiving and storing of the service record includes obfuscating or encrypting the protected content of the service record of the medical imaging device; and
the executing of the executable code stored in the at least one control entry to limit the displaying of protected content includes de-obfuscating or decrypting the protected content of the service record of the medical imaging device only if the protected content is accessible to the user access level.

17. A non-transitory computer readable medium storing instructions readable and executable by at least one computer to:
receive and store as service record of a medical imaging device including: content describing servicing of the medical imaging device, and at least one control entry of the service record of the medical imaging device which comprises executable code; and
view the service record of the medical imaging device including executing the executable code of the at least one control entry to control the view of the service record.

18. The non-transitory computer readable medium of claim 17, wherein the executing of the executable code of the at least one control entry generates processed log data by performing processing of log data generated and stored by the medical imaging device or by invoking an application program to perform the processing of the log data, and displays the processed log data.

19. The non-transitory computer readable medium of claim 17, wherein the executing of the executable code of the at least one control entry controls the medical imaging device to acquire a test image and displays the test image.

20. The non-transitory computer readable medium of claim 17, wherein the executing of the executable code of the at least one control entry limits the view of the service record of the medical imaging device based on a user access level.
